(19) United States
(12) Patent Application Publication
(10) Pub. No.: US 2017/0050054 A1
(43) Pub. Date: Feb. 23, 2017

(54) COSMETIC PRODUCT

(71) Applicant: BEIERSDORF AG, Hamburg (DE)

(72) Inventors: Sven FEY, Hamburg (DE); Dana RETZER, Hamburg (DE); Janne MOEBIUS, Hamburg (DE)

(21) Appl. No.: 15/306,585

(22) PCT Filed: Feb. 5, 2015
(86) PCT No.: PCT/EP2015/052417

§ 371 Date: Oct. 25, 2016

(30) Foreign Application Priority Data
May 6, 2014 (DE) 102014208451.2
May 6, 2014 (DE) 202014003725.6

(51) Int. Cl. A61Q 17/04; A61K 8/66; B05B 11/00; A61K 8/73

(52) U.S. Cl. CPC A61Q 17/04; A61K 8/733; A61K 8/66; B05B 11/0048; B05B 11/0078; B05B 11/3033; B05B 11/3035; A61K 2800/87; A61K 2800/41

(57) ABSTRACT

Cosmetic products, comprising a container having a tubular body, the wall of which has a transmission for visible light of from 25 to about 100%, and on the top end of which a dispenser head is attached, the dispenser head being bounded below by a movable trailing piston, and a cosmetic preparation in the interior of the container which preparation comprises an outer flowable water-based gel-type phase having a transmission for light of 700 nm wavelength of from 30 to 100% and/or a viscosity of 1000-10,000 mPas at a temperature of 25° C. and a shear rate of approximately 10, and an inner solid particulate phase which comprises substantially spherical particles having an average diameter of from 0.1 to 10 mm, the volume ratio of the inner particulate phase to the outer aqueous gel ranging from about 20%:80% to 74%:26%.

The present invention relates to stabilized, oxidation-sensitive and/or UV-sensitive active ingredients, and also to cosmetic and dermatological formulations comprising stabilized oxidation-sensitive and/or UV-sensitive active ingredients.

The present invention relates to cosmetic and/or dermatological dermatological preparations comprising active ingredients for caring for and for protecting the skin, particularly sensitive and dry skin, and also, very especially, skin which is aging or has aged as a result of intrinsic and/or extrinsic factors, and also for supporting the endogenous lipid metabolism of the skin, and also to the use of such active ingredients and combinations of such active ingredients in the field of cosmetic and dermatological skincare.

By cosmetic skincare is meant primarily the strengthening or restoration of the skin's natural function as a barrier against environmental influences (e.g., dirt, chemicals, microorganisms) and against the loss of endogenous substances (e.g., water, natural fats, electrolytes).

Impairment of this function may result in increased absorption of toxic or allergenic substances or to infestation by microorganisms and, consequently, in toxic or allergic skin reactions.

A further aim of skincare is to compensate the loss by the skin of fats and water as a result of everyday washing. This is particularly important when the natural regeneration capacity is not sufficient. Furthermore, skincare products are intended to protect against environmental influences, especially against sun and wind, and to delay skin aging.

Chronological skin aging is caused, for example, by endogenous, genetically determined factors. As a consequence of aging, the epidermis and dermis experience, for example, the following structural damage and functional disorders, which may also be covered by the term “senile xerosis”:
a) dryness, roughness, and development of dryness wrinkles,
b) itching, and
c) reduced refatting by sebaceous glands (e.g., after washing).

Exogenous factors, such as UV light and chemical noxae, may have a cumulative effect and may, for example, accelerate or supplement the endogenous aging processes. In epidermis and dermis, particularly as a result of exogenous factors, for example, the following structural damage and functional disorders occur in the skin, being more far-reaching than the extent and grade of the damage in the case of chronological aging:
d) visible vascular dilations (telangiectasias, couperosis);
e) flaccidity and development of wrinkles;
f) local instances of hyper-, hypo- and dyspigmentation (e.g., age spots), and
g) increased susceptibility to mechanical stress (e.g., cracking).

The present invention relates in particular to products for the care of skin which has undergone natural aging, and also for treating the consequential damage of photoaging, more particularly the phenomena listed under a) to g).

Products for caring for aged skin are known per se. They comprise, for example, retinoids (vitamin A acid and/or its derivatives) or vitamin A and/or its derivatives. Their effect on structural damage, however, is limited in its scope. Moreover, there are considerable difficulties in product development in sufficiently stabilizing the active ingredients against oxidative breakdown. The use of vitamin A acid-containing products, moreover, often gives rise to severe erythematous skin irritation. Retinoids can therefore be used only at low concentrations.

In particular, the present invention relates to cosmetic preparations with effective protection against harmful oxidation processes in the skin, but also for protecting cosmetic preparations themselves, or protecting the constituents of cosmetic preparations, against harmful oxidation processes.

The present invention further relates to antioxidants, preferably those which are used in cosmetic or dermatological skincare preparations. More particularly the invention also relates to cosmetic and dermatological preparations comprising such antioxidants. In one preferred embodiment the present invention relates to cosmetic and dermatological preparations for the prophylaxis and treatment of cosmetic or dermatological changes in the skin, such as skin aging, for example, particularly the skin aging caused by oxidative processes.

The present invention relates, moreover, to active ingredients and preparations comprising such active ingredients for the cosmetic and dermatological treatment or prophylaxis of erythematous, inflammatory, allergic or autoimmunoreactive phenomena, especially dermatoses.

The present invention relates, in another advantageous embodiment, to active ingredient combinations and preparations which are used for the prophylaxis and treatment of photosensitive skin, more particularly of photodermatoses.

The harmful effect of the ultraviolet component of solar radiation on the skin is common knowledge. While rays with a wavelength of less than 290 nm (the so-called UVC region) are absorbed by the ozone layer in the Earth's atmosphere, rays in the range between 290 nm and 320 nm, the so-called UVB range, give rise to erythema, simple sun burn or even burns of greater or lesser severity.

The narrower range around 308 nm is stated as a maximum for the erythema activity of sunlight.

There are numerous compounds known for protecting against UVB radiation, these being derivatives of 3-benzylidenecamphor, of 4-aminobenzoic acid, of cinnamic acid, of salicylic acid, of benzophenone, and also of 2-phenylbenzimidazole.

For the range between about 320 nm and about 400 nm as well, the so-called UVA range, it is important to have filter substances available, since the rays of this range may give rise to reactions in the case of photosensitive skin. It has emerged that UVA radiation causes damage to the elastic and collagenic fibers of the connective tissue, causing the skin to age prematurely, and that it should be regarded as a cause of numerous phototoxic and photoallergic reactions. The damaging influence of the UVB radiation may be intensified by UVA radiation.

For protecting against the rays of the UVA range, therefore, certain derivatives of dibenzoylmethane are used, the photostability of which (Int. J. Cosm. Science 10, 53 (1988)) is not sufficient.

The UV radiation, however, may also lead to photochemical reactions, in which case the photochemical reaction products intervene in the skin's metabolism.

Such photochemical reaction products are primarily radical compounds, examples being hydroxyl radicals. Undefined radical photoproducts which form within the skin itself, as well, may exhibit uncontrolled secondary reactions on account of their high reactivity. But singlet oxygen as well, a nonradical excited state of the oxygen molecule, may occur under UV irradiation, as may short-lived epoxides and many others. Singlet oxygen, for example, is distinguished by increased reactivity relative to the triplet oxygen that is normally present (radical ground state). Nevertheless, excited, reactive (radical) triplet states of the oxygen molecule also exist.

Furthermore, UV radiation is a type of ionizing radiation. There is therefore a risk that ionic species will also form under UV exposure, and then in turn are able to intervene oxidative in the biochemical process.

In order to prevent these reactions, additional antioxidants and/or radical scavengers may be incorporated into the cosmetic and/or dermatological formulations.

Proposals have already been made to use vitamin E, a substance having known antioxidative action, in light protection formulations, although here as well the effect achieved is far behind that hoped for.

It was an object of the invention as well, therefore, to provide cosmetic, dermatological, and pharmaceutical active ingredients and preparations, and also light protection formulations, which serve for prophylaxis and treatment of photosensitive skin, more particularly photodermatoses, preferably PLD.

Other terms for polymorphous photodermatosis are PLD, PLE, Majorca acne and a host of further designations, as reported in the literature (e.g., A. Voelckel et al., Zentralblatt Haut- und Geschlechtskrankheiten (1989), 156, page 2).

Erythematous skin symptoms also occur as accompanying symptoms in certain skin diseases or skin irregularities. For example, the typical skin rash among the symptoms of acne is regularly reddened to a greater or lesser extent.

Antioxidants are used primarily as substances protecting against the deterioration of the preparations in which they are present. Nevertheless, it is known that in human and animal skin as well, unwanted oxidation processes may occur. Such processes play an important role is skin aging.

The essay “Skin Diseases Associated with Oxidative Injury” in “Oxidative Stress in Dermatology”, p. 323 ff. (Marcel Decker Inc., New York, Basel, Hong Kong, editors: Jürgen Fuchs, Frankfurt, and Lester Packer, Berkeley/California) sets out oxidative damage to the skin and its more specific causes.

Also for the reason of preventing such reactions, cosmetic or dermatological formulations may additionally have antioxidants and/or radical scavengers incorporated into them.

Certain antioxidants and radical scavengers are, indeed, known. For instance, US patent specifications 4,144,325 and 4,248,861, along with numerous other documents, have already proposed using vitamin E, a substance with known antioxidative effect, in light protection formulations; nevertheless, here as well, the effect achieved remains a long way behind that hoped for.

It was an object of the present invention, therefore, to find ways to avoid the disadvantages of the prior art. The aim in particular is that the effect of eliminating the damage associated with endogenous, chronological, and exogenous skin aging, and the prophylaxis, should be durable, sustainable, and without risk of side-effects.

Remedying these deficiencies was an object of the present invention.

Known additionally are cosmetic preparations with coenzyme Q-10, from DE-A-33 09 850, which are suitable for the treatment of skin diseases, for the prophylaxis of dystrophic and dysmetabolic states of the skin, and for use with chemical and physical respiration damage or with retarded respiration in conjunction with age and wear.

Coenzyme Q-10 is characterized by the following structural formula

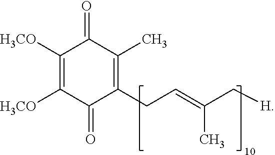

Japanese published specification 58,180,410 describes the suitability of coenzyme Q-10 for cosmetics. It is said to activate the metabolism of skin cells and to suppress oxidation. As a result, coenzyme Q-10 has an important function in preventing skin damage due to UV rays and in preventing skin aging. In 20- to 40-year-olds, the roughness of the skin is meliorated, by the adding of moisture to the skin.

With advantage in accordance with the invention, the quinones may be selected from the group of bioquinones.

Bioquinones are prenylated quinones which occur in the animal and plant kingdoms, where they fulfill biochemical functions. Especially preferred are ubiquinones and plastoquinones.

With advantage in accordance with the invention, the hydroquinones may be selected from the group of the reduced forms of the corresponding bioquinones, in other words, with particular preference, the ubiquinols and plastoquinols.

Ubiquinones represent the most widespread and hence best-investigated bioquinones. Ubiquinones are referred to as Q-1, Q-2, Q-3, etc., according to the number of isoprene units linked in the side chain, or as U-5, U-10, U-15, etc., according to the number of C atoms. They occur preferentially with particular chain lengths, as for example with n=6 in certain microorganisms and yeasts. Q-10 is predominant in the majority of mammals, including humans.

Ubiquinones act as electron transfer agents in the respiratory chain of organisms. They are located in the mitochondria, where they enable the cyclic oxidation and reduction of the substrates in the citric acid cycle.

Plastoquinones have the general structural formula

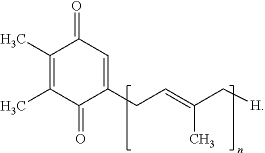

They can be isolated from chloroplasts and they play a part as redox substrates in photosynthesis within cyclic and noncyclic electron transport, being transformed reversibly into the corresponding hydroquinones (plastoquinol). Plastoquinones differ in the number n of the isoprene residues, and are designated accordingly—e.g., PQ-9 (n=9). Other plastoquinones with different substituents on the quinone ring also exist.

One deficiency in the prior art is that ubiquinones and plastoquinones undergo rapid decomposition in an oxidative medium, but also under UV radiation, and in this way lose their activity.

It was an object of the present invention, therefore, to increase the stability of oxidation-sensitive and/or UV-sensitive active ingredients and also to provide stable preparations with oxidation-sensitive and/or UV-sensitive active ingredients, the activity thereof being maintained over a long time.

Liquids may be differentiated in terms of their rheological properties by their flow and deformation characteristics. External forces cause bodies with ideal elasticity to undergo elastic deformation which, when the external force is removed, produces spontaneous, complete reversal of the deformation. Bodies with ideal viscosity are altered irreversibly in their shape by external forces. The increasing deformation is referred to as flow. The majority of liquids have neither ideal viscosity nor ideal elasticity, but instead display both viscous and elastic properties and are therefore referred to as viscoelastic substances.

In the majority of viscoelastic solutions, dispersed particles or gas bubbles will always undergo sedimentation or ascent, respectively. They possess a finite structural relaxation time. This means that the networks in these systems respond to deformation with a corresponding shearing stress. This stress, however, will relax to a value of zero within a finite time, so that the solution as a whole is again in a stable state of rest without stress. This additionally means that these solutions possess a defined zero-shear viscosity and therefore attain a constant viscosity value at low shear rates.

In contrast to these systems, however, there are also systems in which dispersed particles or gas bubbles do not undergo sedimentation. It is notable that these systems flow only above a characteristic value. This value is termed the yield point. On closer examination of the rheological properties of these systems, it is found that in the whole frequency range the storage modulus is independent of the oscillation frequency and is always substantially greater than the loss modulus.

Conversely, even at the smallest frequencies, the complex viscosity does not achieve a constant value, but instead continues to increase.

Carbopol gels contain acrylic acid polymers, which may have a linear or crosslinked construction and which carry a high number of carboxyl groups. In dissolved form, these structures bind water. The neutralization of the carboxyl groups results, on account of their electrostatic repulsion, in expansion and therefore swelling of the polymer chains. In this state, the carbopol gels attain their typical rheological properties such as, for example, increasing the viscosity of the cosmetic preparation and/or developing a yield point.

The effect of the development of a yield point is therefore based on the electrostatic repulsion of the carboxyl groups. Additional electrolytes shield these charges. As a result, the networks collapse, the yield point breaks down, and particles or gas bubbles can no longer be held in suspension.

It was surprising and could not have been foreseen by the skilled person—and herein lies the basis for the achievement of the objects in accordance with the invention—that cosmetic products comprising

a container
which a tubular body,
   whose walls have a transmittance for visible light of 25%-about 100%,
on whose top end a dispenser head is mounted,
which is bounded at the bottom by a movable drawing plunger,
   a cosmetic preparation in the interior of the container that comprises:
an external fluid gellike phase on an aqueous basis
   which has transmittance for light with a wavelength of 700 nm from the range from 30% to 100%
and/or
   which has a viscosity selected from the range of 1000-10 000 mPas at a temperature of 25° C. and a shear rate of approximately 10, preferably of from 3000-9000 at a temperature of 25° C. and a shear rate of approximately 10, especially preferably of 5000-8000 at a temperature of 25° C. and a shear rate of approximately 10,
and
   which optionally comprises customary auxiliaries and or adjuvants dispersible or soluble in water,
an internal solid particulate phase which
   comprises substantially spherical particles whose average diameter is selected from the range from 0.1 to 10 mm, preferably 2 to 7.5 mm,
where the volume ratio of internal particulate phases to external aqueous gel is selected from the range from about 20%:80% to 74%:26%, more preferably 30%:70% to 65%:35%, especially preferably from 40%:60% to 60%:40%,
   remedy the deficiencies of the prior art.

One particularly preferred embodiment of the present invention relates to cosmetic products comprising a container
which a tubular body,
   whose walls have a transmittance for visible light of 25%-about 100%,
on whose top end a dispenser head is mounted,
which is bounded at the bottom by a movable drawing plunger,
   a cosmetic preparation in the interior of the container that comprises:
an external fluid gellike phase on an aqueous basis
   which has a turbidity from the range from 0 to 500, preferably from 0.00001 to 100, especially preferably from 0.01 to 10, and
   which optionally comprises customary auxiliaries and or adjuvants dispersible or soluble in water,
an internal solid particulate phase which
   comprises substantially spherical particles whose average diameter is selected from the range from 0.1 to 10 mm, preferably 2 to 7.5 mm, and where the particulate phase comprises
      a polymer matrix which is substantially insoluble in water,
      one or more substances selected from the group of ubiquinones and/or plastoquinones,
      one or more light filter substances selected from the group of metal oxide pigments,
      optionally one or more light filter substances selected from the group of customary cosmetic light protection filter substances,
where the volume ratio of internal particulate phases to external aqueous gel is selected from the range from about 20%:80% to 74%:26%, more preferably 30%:70% to 65%:35%, especially preferably from 40%:60% to 60%:40%.

The cosmetics products of the invention are from every point of view entirely satisfactory products which are not confined to a limited selection of raw materials.

Accordingly they are especially suitable for serving as a basis for preparation forms having diverse applications.

Preparations advantageous in accordance with the invention may be obtained by virtue of the external fluid gellike phase on an aqueous basis containing one or more hydrocolloids.

“Hydrocolloid” is the shortened technological designation for the inherently more correct term “hydrophilic colloid”. Hydrocolloids are macromolecules which have a largely linear design and possess intermolecular interaction forces which enable secondary and main valence bonds between the individual molecules and hence the formation of a netlike structure. They are partly water-soluble, natural or synthetic polymers which form gels or viscous solutions in aqueous systems. They raise the viscosity of water by either binding water molecules (hydration) or else accommodating and enveloping the water in their interlaced macromolecules, and at the same time restrict the mobility of the water. Water-soluble polymers of this kind constitute a large group of chemically very different natural and synthetic polymers whose common feature is their solubility in water or aqueous media. A requirement for this is that these polymers possess a sufficient number of hydrophilic groups for water-solubility and are not too greatly crosslinked. The hydrophilic groups may be nonionic, anionic or cationic in nature, as for example as follows:

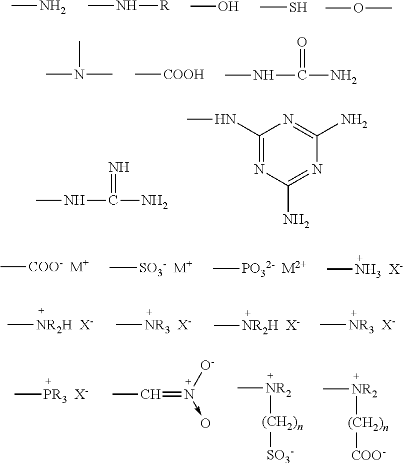

The group of the cosmetically and dermatologically relevant hydrocolloids may be subdivided as follows into:
organic, natural compounds, such as, for example, agar agar, carrageenan, tragacanth, gum Arabic, alginates, pectins, polyoses, guar flour, carob flour, starch, dextrins, gelatin, caseine,
organic modified natural substances, such as, for example, carboxymethylcellulose and other cellulose ethers, hydroethyl- and -propylcellulose and the like,
organic fully synthetic compounds, such as, for example, polyacrylic and polymethacrylic compounds, vinyl polymers, polycarboxylic acids, polyethers, polyimines, polyamides,
inorganic compounds, such as, for example, polysilicas, clay minerals such as montmorillonites, zeolites, silicas.

Hydrocolloids preferred in accordance with the invention are, for example, methylcelluloses, which is the term used for the methyl ethers of cellulose. They are distinguished by the following structural formula

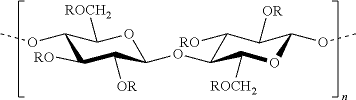

### Structural Formula

in which R may be a hydrogen or a methyl group.

Especially advantageous for the purposes of the present invention are the mixed ethers of cellulose, generally likewise termed methylcelluloses, which as well as a predominant content of methyl groups additionally contain 2-hydroxyethyl, 2-hydroxypropyl or 2-hydroxybutyl groups. Particularly preferred are (hydroxypropyl)methylcelluloses, examples being those obtainable under the commercial designation Methocel E4M from Dow Chemical Comp.

Further advantageous in accordance with the invention is sodium carboxymethylcellulose, the sodium salt of the glycolic acid ether of cellulose, for which R in structural formula I may be a hydrogen and/or CH₂—COONa. Particularly preferred is the sodium carboxymethylcellulose available under the commercial designation Natrosol Plus 330 CS from Aqualon, also referred to as cellulose gum.

Additionally very preferred in the sense of the present invention is xanthan (CAS No. 11138-66-2), also called xanthan gum, which is an anionic heteropolysaccharide which is generally formed by fermentation from corn sugar and is isolated as the potassium salt. It is produced by Xanthomonas campestris and certain other species under aerobic conditions with a molecular weight of 2×10⁶ to 24×10⁶. Xanthan is formed from a chain with β-1,4-bonded glucose (cellulose) with side chains. The structure of the subgroups consists of glucose, mannose, glucuronic acid, acetate, and pyruvate. Xanthan is the name for the first microbial anionic heteropolysaccharide. It is produced by Xanthomonas campestris and certain other species under aerobic conditions with a molecular weight of 2-15 10⁶. Xanthan is formed from a chain with β-1,4-bonded glucose (cellulose) with side chains. The structure of the subgroups consists of glucose, mannose, glucuronic acid, acetate, and pyruvate. The number of pyruvate units determines the viscosity of the xanthan. Xanthan is produced in two-day batch cultures with a yield of 70-90%, based on carbohydrate used. Yields of 25-30 g/l are achieved here. Work-up is accomplished after killing of the culture by precipitation with 2-propanol, for example. Xanthan is subsequently dried and ground.

Another particularly advantageous gel former in the sense of the present invention is carrageen, a gel-forming extract with a construction similar to that of agar, obtained from North Atlantic red algae which belong to the Florideae (Chondrus crispus and Gigartina stellata).

It is common to use the term carrageen for the dry algal product and carrageenan for the extract of that product. The carrageen precipitated from the hot-water extract of the algae is a colorless to sandy colored powder having a molecular weight range of 100 000-800 000 and a sulfate content of about 25%. Carrageenan is very slightly soluble in warm water, and cools to form a thixotropic gel, even if the water content is 95-98%. The double-helix structure of the carrageen is responsible for the strength of the gel. For carrageenan, a distinction is made between three principal constituents: the gel-forming K fraction consists of D-galactose 4-sulfate and 3,6-anhydro-a-D-galactose, which are glycosidically linked alternately in 1,3- and 1,4-positions (agar, in contrast, contains 3,6-anhydro-α-L-galactose). The nongelling λ fraction is composed of 1,3-glycosidically linked D-galactose 2-sulfate and 1,4-bonded D-galactose 2,6-disulfate residues and is slightly soluble in cold water. τ-Carrageenan, which is composed of D-galactose-4-sulfate in 1,3-bonding and 3,6-anhydro-α-D-galactose-2-sulfate in 1,4-bonding, is both water-soluble and gel-forming. Other types of carrageen are likewise designated using Greek letters: α, β, γ, μ, ν, ξ, π, ω, χ. The nature of cations present (K⁺, NH₄⁺, Na⁺, Mg²⁺, Ca²⁺) also influences the solubility of the carrageens.

Polyacrylates are likewise gelators for advantageous use for the purposes of the present invention.

Polyacrylates advantageous in accordance with the invention are acrylate-alkyl acrylate copolymers, especially those selected from the group known as carbomers or carbopols (Carbopol® is in fact a registered trade mark of B.F. Goodrich Company). The acrylate-alkyl acrylate copolymer or copolymers advantageous in accordance with the invention are distinguished in particular by the following structure:

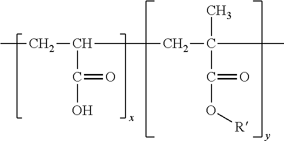

In this formula, R′ is a long-chain alkyl radical and x and y are numbers which symbolize the respective stoichiometric fraction of the comonomers in question.

Particularly preferred in accordance with the invention are acrylate copolymers and/or acrylate-alkyl acrylate copolymers which are available under the trade names Carbopol® 1382, Carbopol® 981, and Carbopol® 5984 from B.F. Goodrich Company.

Further particularly advantageous are copolymers of C₁₀₋₃₀ alkyl acrylates and one or more monomers of acrylic acid, of methacrylic acid or esters thereof, which are crosslinked with an allyl ether of sucrose or with an allyl ether of pentaerythritol.

Particularly advantageous are compounds which carry the INCI name “Acrylates/C 10-30 Alkyl Acrylate Crosspolymer”. Especially advantageous are those obtainable from B.F. Goodrich Company under the trade names Pemulen TR1 and Pemulen TR2.

Further particularly advantageous are ammonium acryloyl dimethyltaurate/vinylpyrrolidone copolymers, more particularly those which have the empirical formula [C₇H₁₆N₂SO₄]ₙ[C₆H₉NO]ₘ, corresponding to a statistical structure as follows

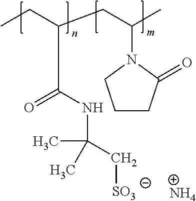

Preferred species in the sense of the present invention are recorded in Chemical Abstracts under the registry numbers 58374-69-9, 13162-05-5, and 88-12-0, and are available under the trade name Aristoflex® AVC from Clariant GmbH.

The total amount of one or more hydrocolloids in the completed cosmetic or dermatological preparations is selected advantageously in accordance with the invention from the range from 0.005 to 5 wt %, preferably between 0.1 and 2.0 wt %, more particularly between 0.25 and 0.75 wt %, based in each case on the total weight of the external fluid gellike phase on an aqueous basis.

The polymer matrix, which constitutes, so to speak, the base structure of the particulate phase, may be selected from the group of customary, cosmetically or dermatologically unobjectionable, substantially water insoluble polymer substances, selected for example from the group of the following substances: algin, carrageen, agar, gellan gum, chitosan.

Especially advantageous is a matrix of alginates, preferably sodium alginates, as are also used in DE 44 24 998 A1.

Metal oxide pigments preferred in accordance with the invention are, in particular, oxides of titanium (TiO₂), of zinc (ZnO), of iron (e.g., Fe₂O₃), of zirconium (ZrO₂), of silicon (SiO₂), of manganese (e.g., MnO), of aluminum (Al₂O₃), of cerium (e.g., Ce₂O₃), mixed oxides of the corresponding metals, and also blends of such oxides, and also the sulfate of barium (BaSO₄).

Especially advantageous is the use of oxides of titanium (TiO₂) as metal oxide pigments particularly preferred in accordance with the invention.

For the purposes of the present invention, advantageously, the pigments may also be employed in the form of commercially available oily or aqueous preliminary dispersions. These preliminary dispersions may advantageously contain added dispersing assistants and/or solubilizers.

With advantage in accordance with the invention, the pigments may have been superficially treated (“coated”), to form or to maintain a hydrophilic, amphiphilic or hydrophobic character, for example. This surface treatment may see the pigments being provided, by conventional methods, with a thin hydrophilic and/or hydrophobic, inorganic and/or organic layer. The various surface coatings may, for the purposes of the present invention, also contain water.

Inorganic surface coatings for the purposes of the present invention may consist of aluminum oxide (Al₂O₃), aluminum hydroxide Al(OH)₃, or aluminum oxide hydrate (also: alumina CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may occur alone, in combination and/or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane having an average chain length of 200 to 350 dimethylsiloxane units and silica gel) or alginic acid. These organic surface coatings may occur alone, in combination and/or in combination with inorganic coating materials.

Zinc oxide particles and preliminary dispersions of zinc oxide particles that are suitable in accordance with the invention are available under the following trade names from the companies listed:

| Trade name | Coating | Manufacturer |
|---|---|---|
| Z- Cote HP1 | 2% Dimethicone | BASF |
| Z- Cote | / | BASF |
| ZnO NDM | 5% Dimethicone | H&R |
| ZnO Neutral | / | H&R |
| MZ 505 M | 5% Methicone | Tayca Corp. |

Suitable titanium dioxide particles and preliminary dispersions of titanium dioxide particles are available under the following trade names from the companies listed:

| Trade name | Coating | Manufacturer |
|---|---|---|
| MT-100TV | Aluminum hydroxide/ | Tayca Corporation |
| | stearic acid | |
| MT-100Z | Aluminum hydroxide/ | Tayca Corporation |
| | stearic acid | |
| Eusolex T-2000 | Alumina/simethicone | Merck KgaA |
| Titandioxid T805 | Octyltrimethylsilane | Degussa |
| (Uvinul TiO₂) | | |
| Tioveil AQ 10PG | Alumina/silica | Solaveil/Uniquema |

A310 Tudor Aspen - Kingfisher Colours Ltd.
Kronos 1171 - Kronos Worldwide, Inc.

Examples of UV filter substances advantageous in accordance with the invention are dibenzoylmethane derivatives, especially 4-(tert-butyl)-4′-methoxydibenzoylmethane (CAS No. 70356-09-1), which is sold by DSM under the brand name Parsol® 1789 and by Merck under the trade name Eusolex® 9020.

Advantageous further UV filter substances for the purposes of the present invention are sulfonated, water-soluble UV filters, such as, for example
phenylene-1,4-bis(2-benzimidazyl)-3,3′-5,5′-tetrasulfonic acid and its salts, particularly the corresponding sodium, potassium or triethanolammonium salts, more particularly the phenylene-1,4-bis(2-benzimidazyl)-3,3′-5,5′-tetrasulfonic acid bis-sodium salt with the INCI name Bisimidazylate (CAS No.: 180898-37-7), which is available for example under the trade name Neo Heliopan AP from Haarmann & Reimer;
salts of 2-phenylbenzimidazole-5-sulfonic acid, such as its sodium, potassium or its triethanolammonium salt, and also the sulfonic acid itself, with the INCI name Phenylbenzimidazole Sulfonic acid (CAS No. 27503-81-7), which is available for example under the trade name Eusolex 232 from Merck or as Neo Heliopan Hydro from Haarmann & Reimer;
1,4-di(2-oxo-10-sulfo-3-bornylidenemethyl)benzene (also: 3,3′-(1,4-phenylenedimethylene)bis(7,7-dimethyl-2-oxobicyclo[2.2.1]hept-1-ylmethanesulfonic acid)) and its salts (especially the corresponding 10-sulfato compounds, more particularly the corresponding sodium, potassium or triethanolammonium salt), which is also called benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic acid (CAS No.: 90457-82-2) and is available for example under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention are, furthermore, those referred to as broad-spectrum filters, i.e., filter substances which absorb both UV-A and UV-B radiation.

Advantageous broad-spectrum filters or UV-B filter substances are, for example, triazine derivatives, such as, for example
2,4-bis{[4-(2-ethylhexyloxy)-2-hydroxy]phenyl}-6-(4-methoxyphenyl)-1,3,5-triazine (INCI: Methylene Bis-Benzotriazolemethylbutylphenol), which is available under the trade name Tinosorb® S from CIBA Chemikalien GmbH;
dioctylbutylamidotriazone (INCI: Diethylhexyl-butamidotriazone), which is available under the trade name UVASORB HEB from Sigma 3V;
tris(2-ethylhexyl) 4,4°,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Octyl Triazone), which is sold by BASF Aktiengesellschaft under the product name UVINUL® T 150.

Another advantageous broad-spectrum filter for the purposes of the present invention is 2,2′-methylenebis(6-(2H-benzotriazol-2-yl)-4-(1,1,3,3-tetramethylbutyl)phenol) (INCI: Bisoctyltriazole), which is available under the trade name Tinosorb® M from CIBA Chemikalien GmbH.

Another advantageous broad-spectrum filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS No. 155633-54-8) with the INCI name Drometrizole Trisiloxane.

The further UV filter substances may be oil-soluble. Examples of advantageous oil-soluble filter substances are as follows:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
derivatives of benzophenone, preferably 2-hydroxy-4-methoxybenzophenone, 2-hydroxy-4-methoxy-4′-methyl-benzophenone, 2,2′-dihydroxy-4-methoxybenzophenone;
and also UV filters bound to polymers.

Another light protection filter substance for use advantageously in accordance with the invention is ethylhexyl 2-cyano-3,3-diphenylacrylate (octocrylene), which is available from BASF under the name Uvinul® N 539.

Particularly advantageous preparations in the sense of the present invention preferably comprise not only the filter substance or substances of the invention but also further UV-A and/or broad-spectrum filters, more particularly dibenzoylmethane derivatives [for example, 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine, in each case individually or in any desired combinations with one another.

The list of the stated UV filters which may be used for the purposes of the present invention is of course not intended to be limiting.

The particulate phase advantageously comprises the substances which absorb UV radiation in the UV-A and/or UV-B range in a total amount of, for example, 0.1 wt % to 30 wt %, preferably 0.5 to 20 wt %, more particularly 1.0 to 15.0 wt %, based in each case on the total weight of the particulate phase.

It is possible and optionally advantageous in accordance with the invention, although not mandatory, for not only the particulate phase but also the external, gel-based fluid phase to be equipped with UV filter substances, provided the turbidity requirements are maintained. Generally speaking, water-soluble UV filter substances are used in the external gel phase.

If water-soluble UV filter substances are to be used in the external gel phase, it contains the substances which absorb UV radiation in the UV-A and/or UV-B range in a total amount of, for example, 0.1 wt % to 30 wt %, preferably 0.5 to 20 wt %, more particularly 1.0 to 15.0 wt %, based in each case on the total weight of the external gel phase.

In order to produce the particles which constitute the internal solid particulate phase it is possible advantageously to use a method which is known from DE 44 24 998 A1. With this method, the composition of which the particulate phase consists, or, optionally, a composition from which the particulate phase is intended to develop, and which is present preferably in fluid form, is shaped to form a jet of liquid which is directed to the environment which brings about hardening and in that the portions are formed by dividing up the jet of liquid, before the environment which brings about hardening, into defined sections in such a way that the sections move on essentially in the direction of the jet of liquid.

This method may be presently embodied by first of all forming, from the composition of which the particulate phase consists, or, optionally, a composition from which the particulate phase is intended to develop, a jet of liquid in which the liquid particles move in the direction of the environment that brings about hardening. After the jet of liquid has been formed, it is divided mechanically into defined sections, with the sections thus formed moving on in the direction dictated by the jet of liquid. The division of the jet of liquid is therefore accomplished by periodic removal of liquid from the jet of liquid, to form the defined sections. The length of these sections may be kept short enough that during the remaining movement up to the environment which brings about hardening, more particularly the surface of a hardening medium, an at least approximate sphere form is formed, by virtue of the surface tension of the liquid medium, so that spherical particles are produced.

In the case of liquid media which have a certain viscosity, it is useful if the liquid medium for forming the jet of liquid is forced through the nozzle. This is customarily accomplished by the application of an excess pressure in the reservoir vessel and/or in the line to the liquid jet nozzle.

In many instances of application, there is a requirement to provide the preferably spherical particle with a coating. Optionally this coating can harden in the same environment, it is possible in accordance with the present invention to coat the formed sections of the liquid medium even prior to hardening, by producing at least one cross-jet of liquid featuring a liquid suitable for enveloping, through which the formed sections pass.

The cross-jet of liquid here may usefully take the form of a flat jet, thereby enlarging the possible area of passage for the sections.

One modification to the method may involve the nozzle arrangement being set up to form at least one jet of liquid, with a movable subdevice for the at least one jet of liquid being arranged below the nozzle arrangement.

This subdevice may be positioned directly at the exit of the nozzle arrangement.

The moving subdevice is preferably drivable in rotation and equipped with elements which interrupt the jet of liquid. Through the rotating drive of the subdevice it is possible to achieve a particularly simple construction of the apparatus according to the invention.

In one advantageous embodiment, the elements which interrupt the jet of liquid may be radially aligned radiant elements in the form of wires, plastic filaments or the like. These elements may be formed without their own dimensional stability, with their radial alignment being obtained only during the rotation of the subdevice.

In another embodiment, a rotating disc may be used which has passage apertures for the jet of liquid, these passage apertures being arranged closely alongside one another over a radius. The webs between the passage apertures in this case form the elements which interrupt the jet of liquid.

The subdevice is usefully surrounded by a cylindrical housing which is open at least in the region of the jet of liquid and that has a catch depression for catching liquid running down from the housing walls. In this way, the liquid spun outward radially by the elements which interrupt the jet of liquid is captured and can be passed back to the associated reservoir vessel. The catch depression here is preferably formed by a circulating groove which is provided with a drain.

In order to form an abovementioned coating on the particles, the apparatus may have a transverse nozzle which is directed at an angle relative to the jet of liquid and which, with regard to the jet of liquid, is arranged downstream from the subdevice. This transverse nozzle is connected to a reservoir vessel for a liquid suitable for enveloping. The transverse nozzle here may be a flat jet nozzle.

In one exemplary embodiment, a liquid jet of a cell emulsion in a sodium alginate solution may emerge from the nozzle, and the transverse jet may be formed by a cell-free sodium alginate solution, while a CaCl₂ solution is located as hardening medium in the catch vessel 5.

As they pass through the cross-jet, which is preferably in the form of a flat jet, the cell-containing liquid sections are enveloped for 3 min with the cell-free alginate solution and are then immediately crosslinked, thereby preventing migration of the cells from the core into the enveloping layer, since the Ca ions diffuse substantially more quickly in the alginate droplets than the cells are able to move.

It is possible for a customary cosmetic container to be equipped with a preparation according to the invention in such a way that the internal particulate phase approaches the densest possible spherical packing (space filling of the cubically densest spherical packing: about 74%) whose cavities are filled out by the external aqueous gel phase.

It is therefore preferred for the volume ratio of internal particulate phase to external aqueous gel to be selected from the range from about 20%:80% to 74%:26%, more preferably 30%:70% to 65%:35%, especially preferably from 40%:60% to 60%:40%.

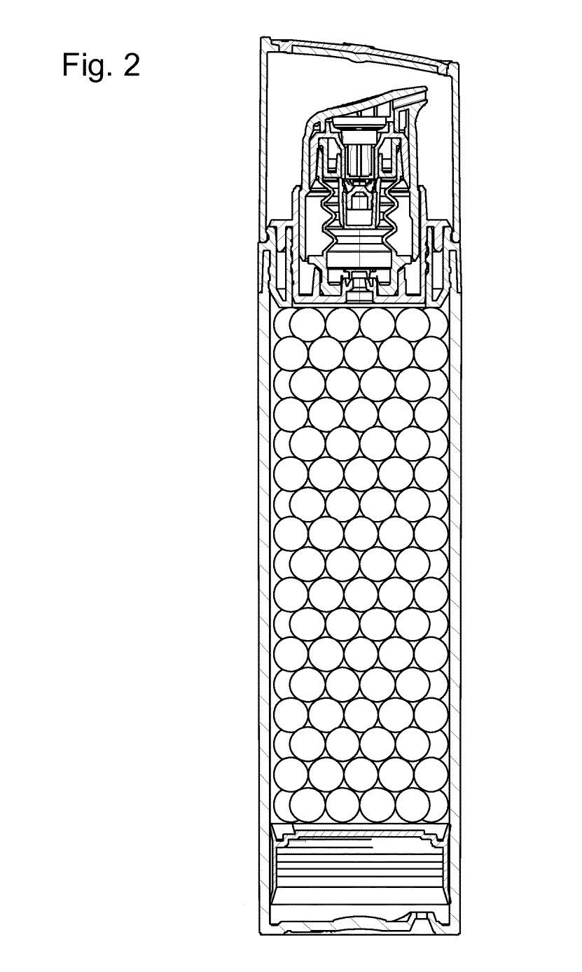

Shown in FIG. 2 is a container which constitutes a customary dispenser with drawing plunger, the container interior being filled with a particulate phase which is surrounded (not shown here) by an external aqueous gel phase.

In the preparations of the invention, the ubiquinone(s) and/or plastoquinone(s) are ideally protected from oxidation and the harmful influence of UV radiation.

When the preparations are applied to a surface, the preparations of the invention can be massaged to form a homogeneous emulsion which goes quickly into the skin and is very appealing in visual and tactile terms.

It is particularly surprising that if a dispenser is used, the shearing forces at the dispenser outlet are enough by themselves to convert those originally in separate compartments (i.e., external gel phase and internal particulate phase) into a visually homogeneous unit.

It is especially advantageous in accordance with the invention if the dispenser, which comprises a container (B)
which a tubular body (K),
   whose walls (Wk) have a transmittance for visible light of 25%-about 100%,
on whose top end a dispenser head (SK) is mounted,
which is bounded at the bottom by a movable drawing plunger (SCH), has a dispenser head (SK)
which has an outer actuating element (BE) having a dispensing aperture (SPO) which is sealed by a nonreturn valve (RV),
a base unit (BO) lying on the tubular body (K) and having an opening (O) to the tubular body, and
a flexible inner, substantially cylindrically shaped hollow body (H) having bellows-like walls (Wh),
   where the elements of the dispenser head are designed such that
the hollow body (H) has openings (Oo) and (Ou) at each of the two end faces (So) and (Su),
the hollow body (H) lies by the bottom end face (Su) on the base unit, with the opening (O) thereof and the bottom opening (Cu) of the hollow body (H) lying over one another,

such that if
the actuating element (BE) as a result of external pressure compresses the inner hollow body (H) in the direction of its longitudinal axis,
the substance located in the interior of the hollow body (H) is conveyed through the nonreturn valve (RV) and the dispenser head (SK) out of the container (B), and
when the external pressure subsides, the recovery force of the flexible inner hollow body (H) draws substance from the tubular body (K) into the flexible inner hollow body (H).

It is especially advantageous if the inner hollow body (H) is designed such that the bottom opening (Ou) at the bottom end face (Su) comprises a means (V) which cuts up the flow of substance from the interior of the tubular body (K) into the hollow body into a plurality of substreams. In the simplest case, a means of this kind consists of a plate having at least two passage openings (Du). With particular advantage these openings may be made radially.

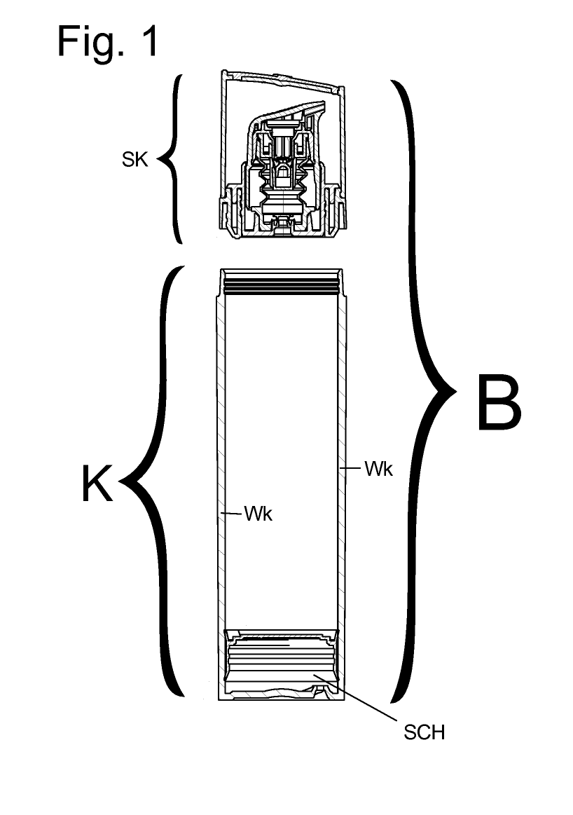

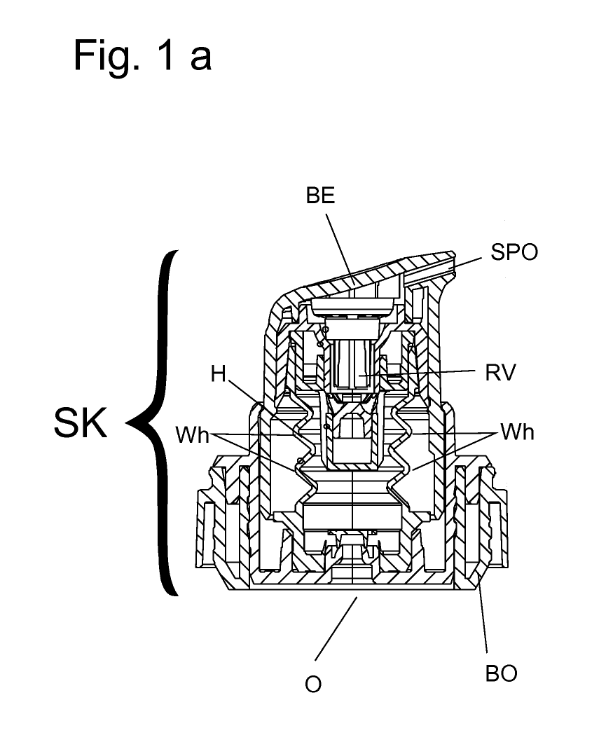

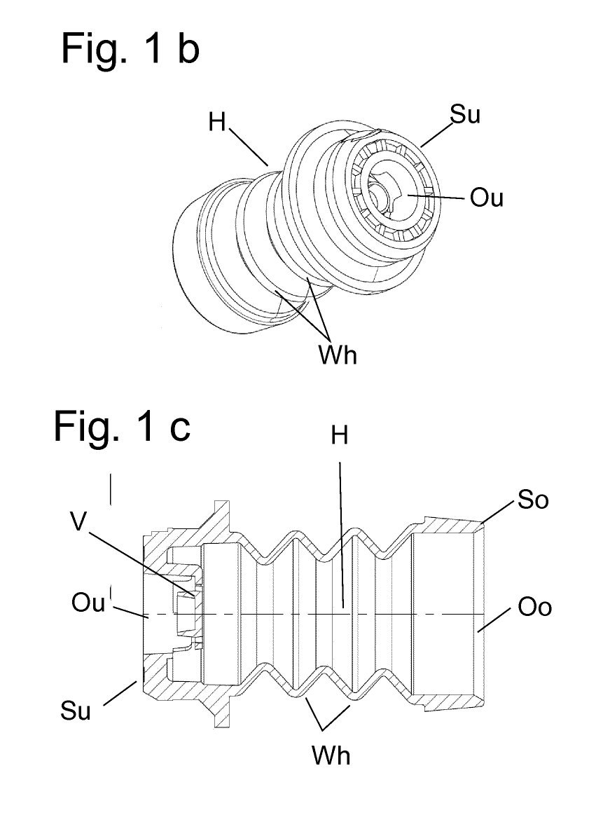

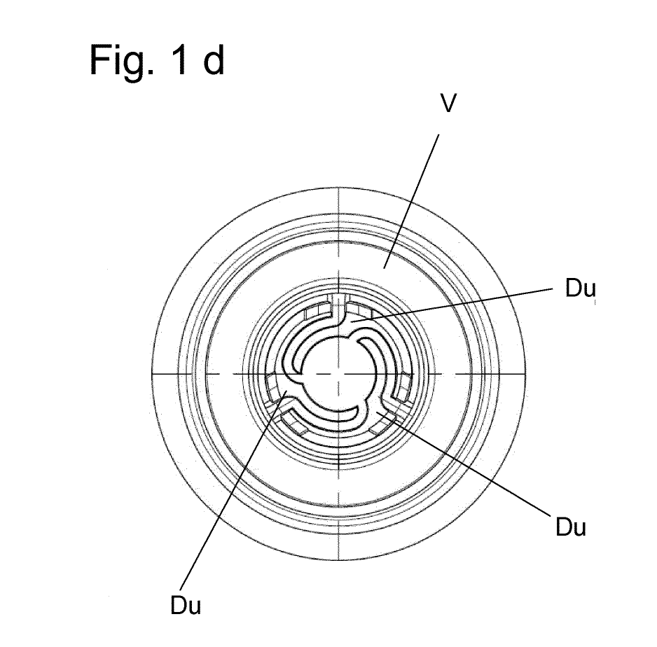

FIG. 1 shows a container (B) which is advantageous in accordance with the invention and which has
a tubular body (K),
at the top end of which a dispenser head (SK) is mounted,
which is bounded at the bottom by a movable drawing plunger (SCH), and
a dispenser head (SK);
   in FIG. 1a, the container (B) being shown separated into body (K) and dispenser head (SK) (in this case provided with an additional closure cap, not shown in any more detail).

FIG. 1a shows a dispenser head (SK)which comprises
an external actuating element (BE) having a dispensing aperture (SPO) which is closed by a nonreturn valve (RV),
a base unit (BO) lying on the tubular body (K) and having an opening (0) to the tubular body, and also
a flexible internal substantially cylindrically shaped hollow body (H) having bellows-like walls (Wh).

FIG. 1b shows a perspective view of one embodiment of the flexible inner substantially cylindrical shaped hollow body (H)
having bellows-like walls (Wh),
where the bottom end face (Su) and its opening (Ou) are visible.

FIG. 1c shows the outline of an embodiment of the flexible inner, substantially cylindrically shaped hollow body (H)
having bellows-like wails (Wh), the elements of the dispenser head being designed such that
   the hollow body (H) has openings (Oo) and (Ou) at each of the two end faces (So) and (Su),
      and where the hollow body (H) is designed such that the bottom opening (Ou) at the bottom end face (Su) comprises a means (V) which cuts up the substance stream from the interior of the tubular body (K) into the hollow body into a plurality of substreams.

FIG. 1d shows a device (V) which is intended for cutting up the substance stream from the interior of the tubular body (K) (not shown here) into the hollow body (H) (not shown here) into three substreams by means of three two passage openings (Du).

The cosmetic and dermatological preprations in accordance with the invention may comprise cosmetic auxiliaries of the kind customarily used in such preparations, examples being preservatives, preserving aids, bactericides, fragrances, substances for preventing foaming, dyes, pigments which have a coloring effect, thickeners, moisturizing and/or humectant substances, fillers which improve the feel on the skin, fats, oils, waxes, or other customary constituents of a cosmetic or dermatological formulation such as alcohols, polyols, polymers, foam stabilizers, electrolytes, organic solvents, or silicone derivatives.

Advantageous preservatives for the purposes of the present invention are, for example, formaldehyde donors (such as, for example, DMDM hydantoin, which is available for example under the trade name Glydant™ from Lonza), iodopropyl butylcarbamates (e.g., those available under the trade names Glycacil-L, Glycacil-S from Lonza and/or Dekaben LMB from Jan Dekker), parabens (i.e., alkyl esters of p-hydroxybenzoic acid, such as methyl, ethyl, propyl and/or butyl paraben), phenoxyethanol, ethanol, benzoic acid, and so on. The preservative system in accordance with the invention customarily further comprises, advantageously, preserving aids as well, such as, for example octoxyglycerol, glycine soya etc.

Particularly advantageous preparations are obtained, moreover, if antioxidants are used as adjuvants or active ingredients. In accordance with the invention the preparations advantageously comprise one or more antioxidants. Antioxidants that are favorable, but whose use is nevertheless optional, may comprise any antioxidants customary or suitable for cosmetic and/or dermatological applications.

With particular advantage for the purposes of the present invention it is possible to use water-soluble antioxidants, such as, for example, vitamins, e.g., ascorbic acid and derivatives thereof, and also D-biotin, natural and/or synthetic isoflavonoids, alpha-glucosylrutin, panthenol, aloe vera, honokiol, and magnolol.

The amount of the antioxidants (one or more compounds) in the preparations is preferably 0.001 to 30 wt %, more preferably 0.05 to 20 wt %, more particularly 0.1 to 10 wt %, based on the total weight of the preparation.

It is especially advantageous if the cosmetic preparations in accordance with the present invention comprise active cosmetic or dermatological ingredients, with preferred active ingredients being antioxidants, which are able to protect the skin from oxidative stress.

Advantageous further active ingredients are natural active ingredients and/or derivatives thereof, such as phytoene, carnitine, carnosine, creatine, N-acetylhydroxyproline, taurine and/or β-alanine, for example.

The external aqueous gel phase of the preparations of the invention may advantageously comprise customary cosmetic auxiliaries, such as, for example, alcohols, especially those of low C number, preferably ethanol and/or isopropanol, diols or polyols of low C number, and also their ethers, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether, and analogous products, polymers, foam stabilizers, electrolytes, and also, in particular, one or more thickeners, which may be selected advantageously from the group of silicon dioxide, aluminum silicates, polysaccharides and/or derivatives thereof, e.g., hyaluronic acid, xanthan gum, hydroxypropylmethylcellulose, particularly advantageously from the group of the polyacrylates, preferably a polyacrylate from the group known as carbopols, examples being carbopols of types 980, 981, 1382, 2984, 5984, in each case individually or in combination. Moisturizers as well may be used with preference.

Moisturizers are compounds or compositions which endow cosmetic or dermatological preparations with the capacity, following application or distribution on the skin surface, to reduce the loss of moisture by the stratum corneum (also called transepidermal water loss (TEWL)) and/or to influence positively the hydration of the stratum corneum.

Advantageous moisturizers for the purposes of the present invention are, for example, glycerol, lactic acid and/or lactates, especially sodium lactate, butylene glycol, propylene glycol, biosaccharide gum-1, Glycine soja, ethylhexyloxyglycerol, pyrrolidonecarboxylic acid, and urea. It is especially advantageous, furthermore, to use polymeric moisturizers from the group of the water-soluble and/or water-swellable and/or water-gellable polysaccharides. Especially advantageous are, for example, hyaluronic acid, chitosan and/or a fucose-rich polysaccharide which is recorded in Chemical Abstracts under the registry number 178463-23-5 and is available for example under the name Fucogel® 1000 from SOLABIA S.A.

The cosmetic or dermatological preparations of the invention may further advantageously—although not mandatorily—comprise fillers, which, for example, further improve the sensory and cosmetic qualities of the formulations and, for example, evoke or reinforce a silky or satiny feel on the skin. Advantageous fillers for the purposes of the present invention are starch and starch derivatives (such as, for example, tapioca starch, distarch phosphate, aluminum and/or sodium starch octenylsuccinate, and the like), pigments which have neither primarily UV filter activity nor coloring activity (such as, for example, boron nitride, etc.) and/or Aerosils® (CAS No. 7631-86-9).

The examples which follow are intended to illustrate the present invention without limiting it. The numerical values in the examples denote weight percentages, based on the total weight of the respective preparations.

### Examples of Internal Particulate Phase:

| | Variants | | | | |
|---|---|---|---|---|---|
| INCI | A | B | C | D | E |
| Algin | 1.5 | 1.5 | 1.5 | 1.5 | 1.5 |
| Ubiquinone | 0.19 | 0.1 | 0.25 | 0.13 | 0.2 |
| CI 77891 | 2.50 | 1 | 0.5 | 2 | 0.3 |
| Cyclomethicone | 10.00 | 1.5 | 5 | 8 | 1.2 |
| (BRB CM 56; | | | | | |
| BRB International) | | | | | |
| Cyclomethicone + | 10.00 | 5 | 15 | 12 | 8 |
| dimethiconol (Xiameter | | | | | |
| PMX 1501 Fluid; | | | | | |
| Dow Corning) | | | | | |
| Sodium stearyl glutamate | 0.25 | 1.1 | — | — | — |
| (Emulgin SG; BASF | | | | | |
| Personal Care and | | | | | |
| Nutrition) | | | | | |
| Polyglyceryl-3 methyl- | — | — | 0.5 | 1 | 2 |
| glucose distearate | | | | | |
| (Tego Care 450; | | | | | |
| Evonik Industries) | | | | | |
| Polymethyl- | 8.00 | 4 | 10 | 8 | 12 |
| silsesquioxane | | | | | |
| (Gransil PSQ; | | | | | |
| Grant Industries) | | | | | |
| Fragrance | 1.00 | 0.49 | 0.6 | 0.8 | 0.4 |
| Phenoxyethanol | 0.60 | 0.6 | 0.6 | 0.6 | 0.6 |
| 1,2-Hexanediol | 0.50 | 0.5 | 0.5 | 0.5 | 0.5 |
| Methylpropanediol | 2.00 | 2 | 2 | 2 | 2 |
| Water | ad 100 | ad 100 | ad 100 | ad 100 | ad 100 |

The formula constituents of examples A to E are processed in analogy to the experimental example in DE 44 24 998, column 5, and shaped into approximately spherical particles with a diameter of about 6 mm

| | Variants | | | | | | | |
|---|---|---|---|---|---|---|---|---|
| | Examples of external aqueous gel: | | | | | | | |
| INCI | 1 | 2 | 3 | 4 | 5 | 6 | 7 | 8 |
| Creatine | 0.04 | 0.07 | 0.04 | 0.07 | 0.04 | 0.07 | 0.04 | 0.07 |
| Creatinine | 0.01 | 0.02 | 0.01 | 0.02 | 0.01 | 0.02 | 0.01 | 0.02 |
| Sodium hyaluronate | 0.2 | 0.4 | 0.2 | 0.4 | 0.2 | 0.4 | 0.2 | 0.4 |
| PEG-40 hydrogenated | 0.5 | 0.8 | 0.5 | 0.8 | 0.5 | 0.8 | 0.5 | 0.8 |
| castor oil (Eumulgin CO | | | | | | | | |
| 40; BASF Personal | | | | | | | | |
| Care and Nutrition) | | | | | | | | |
| Glycerol | 15 | 22 | 15 | 22 | 15 | 22 | 15 | 22 |
| 1,2-Propanediol | 4 | 5 | 4 | 5 | 4 | 5 | 4 | 5 |
| Sodium citrate (trisodium | 1.05 | 1.63 | 1.05 | 1.63 | 1.05 | 1.63 | 1.05 | 1.63 |
| citrate 5,5-hydrate ultrapure | | | | | | | | |
| No. 106431; Merck) | | | | | | | | |
| Sodium hydroxide 45% | 0.8 | 1 | 0.8 | 1 | 0.8 | 1 | 0.8 | 1 |
| Phenoxyethanol | 0.6 | 0.6 | 0.6 | 0.6 | 0.6 | 0.6 | 0.6 | 0.6 |
| 1,2-Hexanediol | 0.5 | 0.5 | 0.5 | 0.5 | 0.5 | 0.5 | 0.5 | 0.5 |
| Methylpropanediol | 2 | 2 | 2 | 2 | 2 | 2 | 2 | 2 |
| Acrylic acid/VP crosspolymer | 1.28 | 2.33 | — | — | — | — | — | — |
| (Ultrathix P-100; Ashland | | | | | | | | |
| Specialty Ingredients) | | | | | | | | |
| Acrylates/C10-30 alkyl | — | — | 0.7 | 1.63 | — | — | — | — |
| acrylate crosspolymer | | | | | | | | |
| Ammonium acryloyldimethyl- | — | — | — | — | 2 | 2.5 | — | — |
| taurate/VP copolymer | | | | | | | | |
| (Aristoflex AVC; Clariant) | | | | | | | | |
| Carbomer (Carbopol 980 or | — | — | — | — | — | — | 0.7 | 1.63 |
| Carbopol 981; Lubrizol | | | | | | | | |
| Advanced Materials) | | | | | | | | |
| Lubrajel Oil Free; Ashland | 4 | 5 | 4 | 5 | 4 | 5 | 4 | 5 |
| Speciality Ingredients | | | | | | | | |
| Water | ad 100 | ad 100 | ad 100 | ad 100 | ad 100 | ad 100 | ad 100 | ad 100 |

The formula constituents of examples 1-8 are stirred with one another at 25° C. until clear gels have formed.

Particles and gels were combined with one another in different volume ratios, as noted in the following combined examples. The first figure shows the particulate phase, the second figure the gel phase

| | 1 | 2 | 3 | 4 | 5 | 6 | 7 | 8 |
|---|---|---|---|---|---|---|---|---|
| A | 25:75 | 30:70 | 50:50 | 40:60 | 25:75 | 40:60 | 40:60 | 50:50 |
| B | 30:70 | 40:60 | 25:75 | 30:70 | 40:60 | 35:65 | 35:65 | 25:75 |
| C | 50:50 | 40:60 | 40:60 | 50:50 | 35:65 | 25:75 | 50:50 | 40:60 |
| D | 40:60 | 35:65 | 35:65 | 35:65 | 30:70 | 40:60 | 30:70 | 50:50 |
| E | 25:75 | 35:65 | 50:50 | 35:65 | 40:60 | 35:65 | 40:60 | 35:65 |

40 ml of each or preparations A1-E8 are dispensed each individually into containers as described in figures FIG. 1a to FIG. 1d.

1.-9. (canceled)

10. A cosmetic product, wherein the product comprises a container and a cosmetic preparation inside the container, the container comprising a tubular body whose walls have a transmittance for visible light of from 25% to about 100% and on whose top end a dispenser head is mounted and which is bounded at its bottom by a movable drawing plunger, and the cosmetic preparation comprising (i) an external fluid gel phase on aqueous basis having a transmittance for light of a wavelength of 700 nm of from 30% to 100% and/or having a viscosity at 25° C. and a shear rate of about 10 of from 1,000 to 10,000 mPas, and optionally comprising customary auxiliaries and or adjuvants which are dispersible or soluble in water, and (ii) an internal solid particulate phase comprising substantially spherical particles whose average diameter is from 0.1 mm to 10 mm, a volume ratio (ii) (i) ranging from about 20%:80% to 74%:26%.

11. The product of claim 10, wherein the volume ratio (ii):(i) ranges from about 30%:70% to 65%: 35%.

12. The product of claim 10, wherein the volume ratio (ii):(i) ranges from about 40%:60% to 60%:40%.

13. The product of claim 10, wherein (i) has a viscosity at 25° C. and a shear rate of about 10 of from 3,000 to 9,000 mPas.

14. The product of claim 10, wherein (i) has a viscosity at 25° C. and a shear rate of about 10 of from 5,000 to 8,000 mPas.

15. The product of claim 10, wherein the average diameter of the substantially spherical particles is from 2 mm to 7.5 mm.

16. The product of claim 10, wherein the dispenser head comprises
an outer actuating element having a dispensing aperture which is sealed by a nonreturn valve,
a base unit lying on the tubular body and having an opening to the tubular body, and
a flexible inner, substantially cylindrically shaped hollow body having bellows-like walls,
elements of the dispenser head being designed such that
the hollow body has openings and at each of its two end faces,
the hollow body lies by a bottom end face on the base unit, with an opening thereof and a bottom opening of the hollow body lying over one another,
such that if
the actuating element as a result of external pressure compresses the inner hollow body in a direction of its longitudinal axis,
a substance inside the hollow body is conveyed through the nonretum valve and the dispenser head out of the container, and
when an external pressure subsides, a recovery force of the flexible inner hollow body draws substance from the tubular body into the flexible inner hollow body.

17. The product of claim 16, wherein the inner hollow body is designed such that its bottom opening at a bottom end face comprises a device which cuts up a flow of substance from an interior of the tubular body into the hollow body into a plurality of substreams.

18. The product of claim 10, wherein (i) comprises one or more hydrocolloids selected from celluloses, methylcelluloses, and polyacrylates.

19. The product of claim 10, wherein (i) comprises one or more hydrocolloids selected from carrageen, xanthan, “Acrylates/C 10-30 Alkyl Acrylate Crosspolymers”, and ammonium acryloyldimethyltaurate/vinylpyrrolidone copolymers.

20. The product of claim 18, wherein a total concentration of the one or more hydrocolloids is less than 1.5 wt %, based on a total weight of the preparation.

21. The product of claim 20, wherein the total concentration of the one or more hydrocolloids is from 0.1 wt % to 1.0 wt %.

22. The product of claim 10, wherein (ii) comprises (A) a polymer matrix which is substantially insoluble in water, (B) one or more substances selected from ubiquinones and/or plastoquinones, (C) one or more light filter substances selected from metal oxide pigments, and, optionally, (D) one or more light filter substances selected from customary cosmetic light protection filter substances.

23. The product of claim 22, wherein (A) is selected from algin, carrageenan, agar, gellan gum, chitosan, and alginates.

24. The product of claim 22, wherein (A) comprises sodium alginate.

25. The product of claim 22, wherein (C) is selected from one or more of TiO₂, ZnO oxides of iron, ZrO₂, SiO₂, oxides of manganese, Al₂O₃, oxides of cerium, mixed oxides of the corresponding metals, and BaSO₄.

26. The product of claim 22, wherein (B) comprises ubiquinone 10 (coenzyme Q10).

27. The product of claim 22, wherein (D) is selected from one or more of octyl methoxycinnamate, octocrylene, Tinosorb M, Tinosorb S, Mexoryl XL, benzophenone-3, benzophenone-4.

28. A cosmetic product, wherein the product comprises a container and a cosmetic preparation inside the container, the container comprising a tubular body whose walls have a transmittance for visible light of from 25% to about 100% and on whose top end a dispenser head is mounted and which is bounded at its bottom by a movable drawing plunger, and the cosmetic preparation comprising (i) an external fluid gel phase on aqueous basis comprising one or more hydrocolloids selected from celluloses, methylcelluloses and polyacrylates and having a transmittance for light of a wavelength of 700 nm of from 30% to 100% and a viscosity at 25° C. and a shear rate of about 10 of from 5,000 to 8,000 mPas, and optionally comprising customary auxiliaries and or adjuvants which are dispersible or soluble in water, and (ii) an internal solid particulate phase comprising substantially spherical particles whose average diameter is from 2 mm to 7.5 mm and comprising (A) a polymer matrix which is substantially insoluble in water, (B) one or more substances selected from ubiquinones and/or plastoquinones, (C) one or more light filter substances selected from metal oxide pigments, and, optionally, (D) one or more light filter substances selected from customary cosmetic light protection filter substances, a volume ratio (ii):(i) ranging from about 60%:40% to 40%:60%.

29. The product of claim 28, wherein the dispenser head comprises
an outer actuating element having a dispensing aperture which is sealed by a nonreturn valve,
a base unit lying on the tubular body and having an opening to the tubular body, and
a flexible inner, substantially cylindrically shaped hollow body having bellows-like walls,
elements of the dispenser head being designed such that
the hollow body has openings and at each of its two end faces,
the hollow body lies by a bottom end face on the base unit, with an opening thereof and a bottom opening of the hollow body lying over one another,
such that if
the actuating element as a result of external pressure compresses the inner hollow body in a direction of its longitudinal axis,
a substance inside the hollow body (H) is conveyed through the nonretum valve and the dispenser head out of the container, and
when an external pressure subsides, a recovery force of the flexible inner hollow body draws substance from the tubular body into the flexible inner hollow body.
